(19) United States
(12) Patent Application Publication
(10) Pub. No.: US 2025/0241708 A1
(43) Pub. Date: Jul. 31, 2025

(54) SURGICAL DEVICES AND METHODS FOR HARVESTING A GRAFT FROM A TENDON

(71) Applicant: Relign Corporation, Campbell, CA (US)

(72) Inventors: Aaron Germain, San Jose, CA (US); Kevin Christopher Felt, Morgan Hill, CA (US)

(21) Appl. No.: 19/036,413

(22) Filed: Jan. 24, 2025

Related U.S. Application Data
(60) Provisional application No. 63/626,215, filed on Jan. 29, 2024.

(51) Int. Cl. A61B 18/16; A61B 18/00; A61B 18/12; A61B 18/14; A61B 90/00

(52) U.S. Cl. CPC A61B 18/16; A61B 18/148; A61B 90/08; A61B 90/361; A61B 2018/00059; A61B 2018/00202; A61B 2018/00601; A61B 2018/126; A61B 2018/1407; A61B 2018/162; A61B 2090/0807

(57) ABSTRACT

An electrosurgical device for harvesting a graft of a tendon is discussed and illustrated variously herein. The device can include a housing having an elongate shape, the housing configured for insertion into an incision of a patient and is configured to contact the tendon upon insertion. The device can include an actuation element moveable relative to the housing. The device can include an electrode coupled to the actuation element for movement therewith, the electrode is configured to cut the tendon using radiofrequency (RF) energy.

### CLAIM OF PRIORITY

This application claims the benefit of U.S. Provisional Patent Application Ser. No. 63/626,215, filed on Jan. 29, 2024, the benefit of priority of which is claimed hereby, and which is incorporated by reference herein in its entirety.

### FIELD OF THE INVENTION

The present invention relates generally to medical devices and to surgical procedures using an arthroscopic device for harvesting a portion of a tendon such as for use in anterior cruciate ligament (“ACL”) reconstruction.

### BACKGROUND

ACL and other ligament injuries have become increasingly common for both athletes and non-athletes. ACL reconstruction is commonly performed to replace an injured ACL. The goal of ACL reconstruction is to restore knee stability and function. Autografts and allografts are the two primary types of grafts used in ACL reconstruction. Autografts involve the use of the patient's own tissue, while allografts use tissue from a cadaver. Autografts are often preferred due to their lower risk of rejection and better integration with the patient's body.

The most commonly used tissues for autografts in ACL reconstruction are the hamstring tendon, patellar tendon, and the quadriceps tendon. Each of these options has its own advantages and disadvantages. The quadriceps tendon is desirable as it provides for good thickness, high collagen content, and lower incidence of harvest site pain. However, the harvesting of the quadriceps tendon can be technically challenging and may require multiple tools, which can complicate the procedure and extend the surgery time. Current surgical systems used for the harvesting of the quadriceps tendon are complex and involve multiple different surgical tools. These systems rely on mechanical cutting devices that can result in uneven cuts and fraying of the graft. Uneven cutting and/or fraying requires additional trimming of the tendon graft resulting in extended surgery time and complexity.

### OVERVIEW

There is a need for more simplified, efficient, and precise surgical devices that can be used for harvesting quadriceps tendon autografts. There is also a need for surgical devices that minimize tissue damage, reduce the number of tools required, and improve the overall outcomes of ACL reconstruction surgeries. The electrosurgical devices (sometimes simply referred to as surgical devices or devices) discussed herein can be inserted into the patient such as via a small minimally invasive incision. The present devices are configured for radiofrequency (“RF”) ablation of the tendon to cut an autograft from the tendon, which allows for enhanced precision and reduced tissue damage as further discussed herein. The surgical devices disclosed herein can include further capabilities such as the ability to perform imagining functions to visualize RF cutting using one or more cameras and the ability to harvest bone from the patella.

The present inventors contemplate the surgical devices disclosed herein can reduce costs by eliminating multiple surgical tools, reduce patient discomfort, reduce surgical complexity and reduce surgical time among other benefits. Additionally, the present inventors realize that the surgical devices disclosed herein provide for various additional benefits including enhanced precision, direct visualization by the surgeon, customizable graft size, reduced tissue damage, ability to conduct bone graft harvesting and improved ease of use. The present inventors also contemplated that the devices disclosed herein can be configured as disposable single use devices or can be configured as reusable sterilizable devices.

Regarding the simplification of the procedure, the present devices integrate multiple functions into a single tool, reducing the number of instruments required for the harvesting process. Traditional methods may require multiple different tools, while the present devices simplify the instrumentation, making the surgical procedure more efficient and less cumbersome.

Regarding the enhanced precision provided by the present devices, the present devices can be a bipolar electrosurgical resection apparatuses. Such a configuration allows for more precise cutting compared to mechanical cutting tools. This precision can result in a more uniform graft and minimize the need for subsequent trimming, which can lead to a better quality graft and improve surgical outcomes. The present devices use of RF energy for cutting allows for plasma formation and vaporization of tissue, which can result in cleaner cuts and less tissue damage compared to mechanical blades.

The present devices can be equipped with one or more cameras, visual indicia such as LEDs and/or the option to integrate the devices with a reusable endoscope. These features can provide surgeons with visualization of device location and/or visualization of the tendon during the harvesting process. These features can help ensure accurate placement and cutting of the tendon, reducing the risk of error.

The present devices can allow for precise control over the longitudinal and lateral dimensions of the harvested graft. Surgeons can use the visual LEDs to precisely gauge and control the length of the graft and the electrode can be a loop or can be otherwise shaped to control a lateral dimension such as the diameter of the graft. This enables precision and customization of the graft size to the specific needs of the patient.

The present devices can be configured to harvest bone from the patella. For example, the surgical device can harvest a bone plug from the patella, which can be used for bone-to-bone grafting. This feature can enhance the strength of the graft and promote better integration into the recipient site.

The present surgical devices can be configured to be hand-actuated or motor-driven, offering flexibility in operation. Additionally, the design can include features such as retractable electrodes and position indicating visual LEDs, which aid in the ease of use during surgery.

The following, non-limiting examples, detail certain aspects of the present subject matter to solve the challenges and provide the benefits discussed herein, among others.

Example 1 is an electrosurgical device for harvesting a graft of a tendon optionally comprising: a housing having an elongate shape, the housing configured for insertion into an incision of a patient and is configured to contact the tendon upon insertion; an actuation element moveable relative to the housing; and an electrode coupled to the actuation element for movement therewith, wherein the electrode is configured to cut the tendon using radiofrequency (RF) energy.

In Example 2, the subject matter of Example 1 optionally includes, wherein the electrosurgical device is a bipolar electrosurgical device with the electrode configured as an active electrode and the housing is configured as a return electrode, and wherein the return electrode serves as a return path for RF current.

In Example 3, the subject matter of Examples 1-2 optionally includes, wherein the electrode is configured as a loop electrode, and wherein the loop electrode is sized to cut the tendon to a desired lateral dimension.

In Example 4, the subject matter of Examples 1-3 optionally includes, wherein the electrode is a shape memory material and is configured to assume a pre-defined shape upon deployment to cut the tendon.

In Example 5, the subject matter of Examples 1-4 optionally includes, a plurality of light emitting diodes (LEDs), wherein the plurality of LEDs are position indictive of various desired longitudinal lengths for the graft, wherein the plurality of LEDs are configured to produce illumination visible through skin of the patient when the housing is inserted into the incision of the patient.

In Example 6, the subject matter of Example 5 optionally includes, at least another LED coupled for movement with the electrode, wherein the at least another LED is indicative of the position of the electrode and produces illumination visible through the skin of the patient when the housing is inserted into the incision of the patient.

In Example 7, the subject matter of Examples 1-6 optionally includes, wherein the electrode is protected by at least one of the housing and the actuation element during initial insertion of the housing into the incision, and wherein the actuation element is configured to actuate the electrode to a plunge position external of the actuation element and the housing once inserted to a desired position.

In Example 8, the subject matter of Example 7 optionally includes, wherein the actuation element is configured to actuate the electrode by at least one of linear translation and rotation of the electrode relative to the housing and a distal shaft of the actuation element.

In Example 9, the subject matter of Examples 1-8 optionally includes, one or more cameras coupled to the actuation element, wherein the one or more cameras are positioned with a field of view that includes the electrode and at least a portion of the tendon cut by the electrode.

In Example 10, the subject matter of Examples 1-9 optionally includes, a bone cutting tool configured to harvest a portion of a bone connected to the graft.

In Example 11, the subject matter of Example 10 optionally includes, wherein the bone cutting tool includes a bone corer and a center drill configured to retain the portion of the bone for positioning and preventing drift during harvesting of the portion of the bone.

In Example 12, the subject matter of Examples 1-11 optionally includes, wherein the actuation element is configured to couple with a second device for one of visualization of the electrode or to drive actuation of the actuation element.

Example 13 is a method of harvesting a graft from a quadriceps tendon using a surgical device, optionally comprising: accessing the quadriceps tendon of a patient; passing the surgical device into a contacting position along a portion of the quadriceps tendon; positioning the surgical device along the portion of the quadriceps tendon to achieve a desired longitudinal length of the graft by illuminating one or more portions of the surgical device when inserted into the patient in the contacting position; and actuating an electrode to cut the graft using radiofrequency (RF) energy.

In Example 14, the subject matter of Example 13 optionally includes, visualizing the electrode with one or more cameras positioned adjacent the electrode.

In Example 15, the subject matter of Examples 13-14 optionally includes, wherein actuating the electrode includes deploying the electrode from an initial insertion position where the electrode is protected by at least one of a housing or an actuation shaft of the surgical device.

In Example 16, the subject matter of Example 15 optionally includes, wherein actuating the electrode includes at least one of linear translation and rotation of the electrode relative to the housing or the actuation shaft.

In Example 17, the subject matter of Examples 13-16 optionally includes, harvesting a portion of a patella with a bone cutting tool of the surgical device, wherein the patella is connected to the graft.

In Example 18, the subject matter of Examples 13-17 optionally includes, wherein positioning the surgical device to achieve the desired longitudinal length of the graft includes providing a proximal portion of the surgical device with a plurality of light emitting diodes (LEDs) coupled thereto, wherein the plurality of LEDs are position indictive of various possible desired longitudinal lengths for the graft, wherein the plurality of LEDs are configured to produce illumination visible through skin of the patient when the surgical device is inserted into the patient.

In Example 19, the subject matter of Example 18 optionally includes, wherein positioning the surgical device to achieve the desired longitudinal length of the graft includes using one or more LEDs coupled for movement with the electrode, wherein the one or more LEDs are indicative of the position of the electrode and produce illumination visible through the skin of the patient when the surgical device is inserted into the patient.

Example 20 is a bipolar electrosurgical device for harvesting a graft of a tendon optionally comprising: a return electrode configured to contact the tendon, wherein the return electrode is configured for insertion into an incision of a patient and is configured to contact the tendon upon insertion to provide a return path for radiofrequency (RF) current; an actuation element moveable relative to the return electrode; and an active electrode coupled to the actuation element for movement therewith, wherein the active electrode is configured to cut the tendon using the RF current.

In Example 21, the subject matter of Example 20 optionally includes, wherein the active electrode is configured as a loop electrode, and wherein the loop electrode is sized to cut the tendon to a desired diameter.

In Example 22, the subject matter of Examples 20-21 optionally includes, wherein the active electrode is a shape memory material and is configured to assume a pre-defined shape upon deployment from one of the actuation element or the return electrode.

In Example 23, the subject matter of Examples 20-22 optionally includes, wherein the return electrode includes a plurality of light emitting diodes (LEDs) coupled thereto, wherein the plurality of LEDs are position indictive of various desired longitudinal lengths for the graft, wherein the plurality of LEDs are configured to produce illumination visible through skin of the patient when a portion of the bipolar electrosurgical device is inserted into the incision of the patient.

In Example 24, the subject matter of Example 23 optionally includes, a colored LED coupled to the actuation element adjacent the active electrode, wherein the colored LED is indicative of the position of the active electrode and produces illumination visible through the skin of the patient when the portion of the bipolar electrosurgical device is inserted into the incision of the patient.

In Example 25, the subject matter of Examples 20-24 optionally includes, wherein the active electrode is protected by at least one of the return electrode and the actuation element during initial insertion of the return electrode into the incision, and wherein the actuation element is configured to actuate the active electrode to a plunge position external of the actuation element and the return electrode once inserted to a desired position.

In Example 26, the subject matter of Example 25 optionally includes, wherein the actuation element is configured to actuate the active electrode by at least one of linear translation and rotation of the active electrode relative to the return electrode and a distal shaft of the actuation element.

In Example 27, the subject matter of Examples 20-26 optionally includes, one or more cameras coupled to the actuation element, wherein the one or more cameras are positioned with a field of view that includes the active electrode and at least a portion of the tendon cut by the active electrode.

In Example 28, the subject matter of Examples 20-27 optionally includes, a bone cutting tool configured to harvest a portion of a bone connected to the graft, wherein the bone cutting tool includes a bone corer and a center drill configured to retain the portion of the bone for positioning and preventing drift during harvesting of the portion of the bone.

In Example 29, the subject matter of Examples 20-28 optionally includes, wherein the actuation element is configured to couple with a second device for one of visualization of the active electrode or to drive actuation of the actuation element.

Example 30 is an apparatus comprising means to implement of any of Examples 1-29.

Example 31 is a system to implement of any of Examples 1-29.

Example 32 is a method to implement of any of Examples 1-29.

In Example 33, the instruments, methods or systems of any one or any combination of Examples 1-29 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1A is a perspective view of a surgical device configured for harvesting a graft from a quadriceps tendon according to an example of the present disclosure.

FIG. 1B is a plan view of a lateral side of the surgical device of FIG. 1A.

FIG. 1C is a proximal side view of the surgical device of FIGS. 1A and 1B.

FIG. 1D is an enlarged perspective view of a distal end portion of the surgical device of FIGS. 1A and 1B.

FIG. 2A is a perspective view of the surgical device of FIGS. 1A-1D with an active electrode actuated to an initial plunge position according to an example of the present disclosure.

FIG. 2B is an enlarged perspective view of the distal end portion of the surgical device of FIG. 2A showing the active electrode and other components according to an example of the present disclosure.

FIG. 2C is a proximal side view of the surgical device in the initial plunge configuration of FIGS. 2A and 2B.

FIG. 3 is a perspective view of the surgical device of FIGS. 1A-2B with an active electrode actuated and moved to a completed tendon cutting position according to an example of the present disclosure.

FIG. 3A is a plan view of an ex vivo facing side of the surgical device of FIG. 3 in the tendon cutting configuration according to an example of the present disclosure.

FIG. 4 is a perspective view of a surgical system that includes another surgical device configured for harvesting a graft from a quadriceps tendon according to another example and an endoscope.

FIG. 4A is an enlarged view of the distal end portion of the surgical device of FIG. 4 according to another example of the present disclosure.

FIG. 5 is a perspective view of yet another surgical device configured for harvesting a graft from the quadriceps tendon according to an example of the present disclosure.

FIG. 6 is a perspective view the surgical device of FIG. 5 with an active electrode captured in a moveable shaft in an insertion position according to an example of the present disclosure.

FIG. 7 is a perspective view of the surgical device of FIG. 5 with the active electrode moved to the initial plunge position according to an example of the present disclosure.

FIG. 8 is a perspective view of the surgical device of FIG. 5 with the active electrode actuated and moved to a completed tendon cutting position according to an example of the present disclosure.

FIG. 9 is a perspective view of another surgical device configured for harvesting a graft from a quadriceps tendon and a bone graft from a patella according to an example of the present disclosure.

FIG. 10 is a perspective view of the surgical device of FIG. 9 with an active electrode moved to the initial plunge position according to an example of the present disclosure.

FIG. 10A is a plan view of a lateral side of the surgical device of FIG. 10.

FIG. 10B is a plan view from a distal side of the surgical device of FIGS. 10 and 10A.

FIG. 11 is a perspective view of the surgical device of FIG. 9 with the active electrode actuated and moved to a completed tendon cutting position and a bone cutting tool moved to a completed bone cutting position according to an example of the present disclosure.

FIGS. 12-14 are cross-sectional views of a portion of the surgical device of FIG. 9 illustrating aspects and movement of components of the bone cutting tool according to an example of the present disclosure.

FIG. 15 is a schematic view of a knee joint with the graft from the quadriceps tendon and the bone graft from the patella resulting from the surgical device of FIGS. 9-14 illustrated according to an example of the present disclosure.

FIG. 16 is a perspective view of another surgical device that can be motor driven and configured for harvesting a graft from a quadriceps tendon.

FIG. 17 is a flow chart of a method for performing an autograft using a surgical device according to an example of the present application.

### DETAILED DESCRIPTION OF THE INVENTION

The present invention relates to surgical devices for harvesting a graft from a tendon of the body. Although described in reference to harvesting an autograft from a quadriceps tendon, the surgical devices discussed herein can be used for harvesting of other tendon(s) from other joints. Thus, the surgical devices are not limited to harvesting of the graft from the quadriceps tendon or to ACL reconstruction surgery. Several embodiments of the surgical devices will now be described to provide an overall understanding of the principles of the form, function and methods of use. In general, the present disclosure provides for electrosurgical devices that can be used as surgical devices for harvesting of a tendon using RF energy. However, the electrosurgical devices disclosed can perform more than one surgical function. Thus, the electrosurgical devices can be configured for anatomy visualization and/or cutting and harvesting bone in addition to or in alternative to harvesting of the tendon, for example. The surgical devices can be entirely disposable, can be entirely sterilizable for reuse or can be an assembly with a disposable probe portion configured for detachable coupling to a non-disposable reusable handpiece. This description of the general principles of this invention is not meant to limit the inventive concepts in the appended claims.

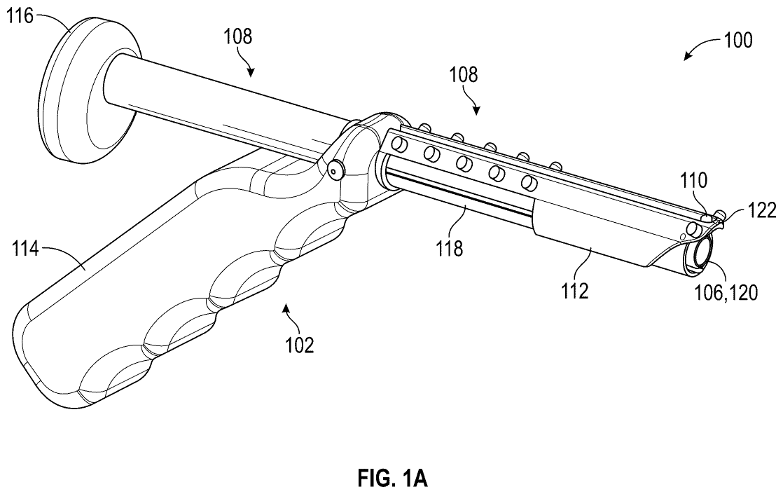

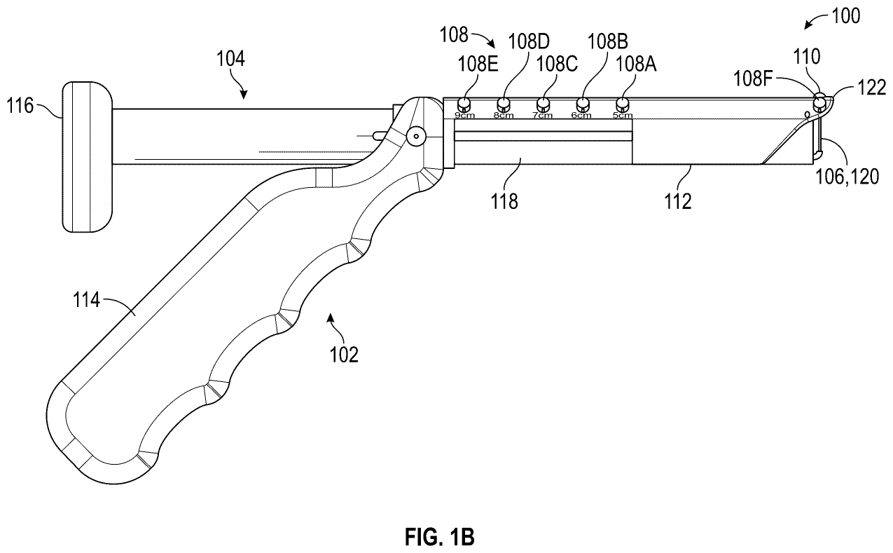

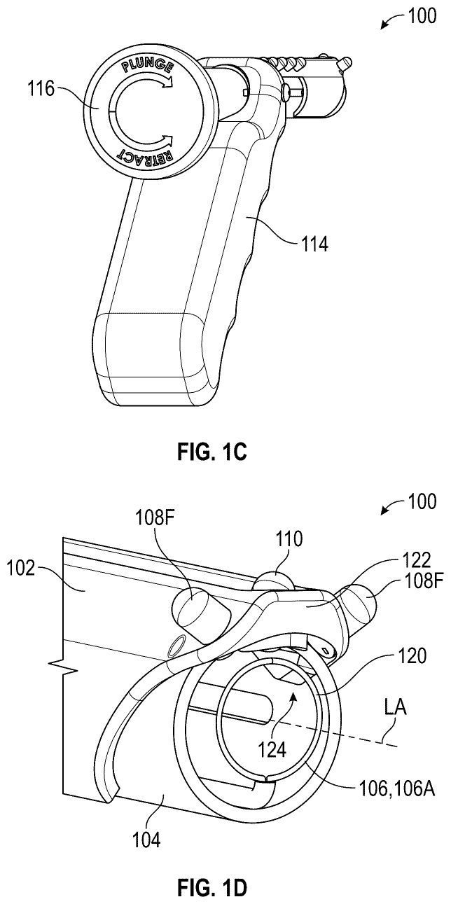

FIGS. 1A-1D show an surgical device 100. As shown in FIGS. 1A and 1B, the surgical device 100 can include a housing 102, an actuation element 104, an electrode 106, a first plurality of light emitting diodes (LEDs) 108 and a second one or more LEDs 110. The housing 102 can include a return electrode 112 and a handpiece 114. The actuation element 104 can include a handle 116 and a shaft 118. The electrode 106 can be an active electrode 120 (FIGS. 1A, 1B and 1D).

The housing 102 can comprise a stator portion of the surgical device 100 and can have a portion with an elongate longitudinal length. The housing 102 can include a central recess or lumen and can have an open frame or tubular type construction configured to receive the actuation element 104, in particular, the elongate shaft 118 therein. The housing 102 can include a distal end 122 and other distal portions configured for insertion into a patient and a proximal end and proximal portions (e.g., the handpiece 114). The handpiece 114 can be external to an incision of the patient during operation of the surgical device 100 and can be used for grasping, positioning and retaining the surgical device 100. The housing 102, in particular the return electrode 112, can be configured to contact (e.g., abut) the tendon upon insertion into an incision of the patient to provide for a return path for RF current applied to the tendon by the active electrode 120.

The actuation element 104, in particular the shaft 118, can be telescopically received by the housing 102. The actuation element 104 can include tube(s) or outer sleeve(s) to receive components such as wires, additional shafts, and the like and allow such components to pass therethrough. The actuation element 104 can be coupled to the electrode 106 (the active electrode 120). The actuation element 104 can be moveable (e.g., linearly translatable and/or rotatable) relative to the housing 102, which can be stator. The handle 116 can be rotatable relative to the shaft 118. Thus, the handle 116 can be coupled to the electrode 106 for actuating rotation of the electrode 106 as further described herein. The shaft 118 can be coupled to the electrode 106 to drive linear translation of the electrode 106 relative to the housing 102 as further illustrated and described. The handle 116 can be external to the incision of the patient during operation of the surgical device 100 and can be used for manipulation of the actuation element 104 (the shaft 118) and the electrode 106 as further described herein.

According to one example, the first plurality of LEDs 108 can be coupled to the housing 102 along a portion of the housing 102 configured for insertion into the patient. However, the first plurality of LEDs 108 can be positioned on an ex vivo facing portion of the housing 102. The first plurality of LEDs 108 can be adjacent or at a proximal portion of the housing 102 and can additionally be at or adjacent the distal end 122. The first plurality of LEDs 108 can be arranged in a series of desired increments. For example, at least some of the plurality of LEDs 108 (e.g., LEDs 108A, 108B, 108C, 108D and 108E as shown in FIG. 1B) can be arranged to be position indictive of various desired longitudinal lengths for the graft (e.g., having particular distances corresponding to typical graft lengths such as 5 mm, 6 mm, 7 mm, 8 mm or 9 mm). The first plurality of LEDs 108 can also include a distal tip LED(s) 108F as shown in FIG. 1B. The first plurality of LEDs 108 can be configured to produce illumination visible through skin of the patient when the surgical device 100, in particular the parts of the housing 102, are inserted into the incision of the patient.

The second one or more LEDs 110 can be coupled for movement with the electrode 106. In particular, the second one or more LEDs 110 can be coupled to the actuation element 104 (e.g., the shaft 118) adjacent to and approximating the location of the electrode 106. The second one or more LEDs 110 can be along an ex vivo facing portion of the shaft 118. The second one or more LEDs 110 can include at least one colored LED (e.g., red, green, or blue). The second one or more LEDs 110 can be indicative of the position of the electrode 106 and can be configured to produce illumination visible through the skin of the patient when the housing 102 is inserted into the incision of the patient.

The first plurality of LEDs 108 and/or the second one or more LEDs 110 can provide surgeon with direct visualized location of where the distal portions of surgical device 100 are located within the patient. The surgeon can use the first plurality of LEDs 108 and/or the second one or more LEDs 110 to gauge insertion distance of the surgical device 100 into the patient for how long a length of the quadriceps tendon is being harvested. As discussed, the second one or more LEDs 110 can be attached to and/or can approximate the electrode 106. Thus, the second one or more LEDs 110 can move with the electrode 106 during actuation thereof back proximally toward the first plurality of LEDs 108A, 108B, 108C, 108D and 108E (FIG. 1B) allowing the surgeon to judge the length of the graft being harvested. The second one or more LEDs 110 can be aligned with one of the first plurality of LEDs 108A, 108B, 108C, 108D and 108E (FIG. 1B) to obtain the desired length of tendon graft precisely. Use of the first plurality of LEDs 108 and/or the second one or more LEDs 110 can allow for more exact repetition, with more accurate graft length results and allows for surgeon visualization of the surgical device 100 through the patient's skin during the harvesting process.

FIG. 1C shows the surgical device 100 from the proximal side particularly showing the handpiece 114 and the handle 116 that are graspable by the surgeon external to the incision. The handle 116 as described previously and further described subsequently can be rotatable by the surgeon to retract or deploy the electrode 106 (FIGS. 1A and 1B) within the patient.

FIG. 1D is an enlarged view of the distal end 122 of the housing 102 with the electrode 106 in a retracted insertion position. The second one or more LEDs 110 are shown approximating a location of the electrode 106 and the LEDs 108F are shown approximating the location of the distal end 122. The electrode 106 can be protected by at least one of the housing 102 and the actuation element 104 in the insertion position illustrated. This insertion position can be used during initial insertion of the housing 102, the shaft 118 and the electrode 106 into the incision. According to the example of FIG. 1D, the electrode 106 is configured as a loop electrode 106A. The electrode 106 has a ring shape. The loop electrode 106A is sized to cut the tendon to a desired diameter. However, according to other examples, the loop electrode 106A can be rectangular, hexagonal or otherwise shaped as desired to provide for a desired cross-sectional shape for the graft (e.g., to provide for a desired lateral geometric width and/or thickness for the graft).

As shown in FIG. 1D, the surgical device 100 can include one or more cameras 124 coupled to at least one of the actuation element 104 and the housing 102. The one or more cameras 124 can be adjacent the distal end 122 when the electrode 106 is in the insertion position. The one or more cameras 124 can travel with linear movement of the electrode 106. According to one example, the one or more cameras 124 can be an on-chip controlled device(s) with the chip being proximate the one or more cameras 124 or on the surgical device at another location. For example, the one or more cameras 124 can utilize Complementary Metal-Oxide Semiconductor Active Pixel Sensors (CMOS-APS). The CMOS-APS are configured for sensing at an infrared wavelength range, a visual light wavelength range or another wavelength range as desired. Optionally, a light source (not shown) can be used with the one or more cameras 124. The one or more cameras 124 can be angled relative to a longitudinal axis LA of the actuation element 104 and the housing 102 so as to be pointed toward the longitudinal axis LA and axially along the longitudinal axis LA. Due to such angulation of the one or more cameras 124, the one or more cameras 124 can have a field of view that includes the electrode 106 and at least portion of the tendon cut by the electrode 106. However, according to further examples the one or more cameras 124 and/or associated light sources can be otherwise angulated so as to cast light and view in any direction(s) as desired. For example, target tissue that has yet to be cut can be visualized before and during cutting. Thus, the one or more cameras 124 can be positioned as desired. This can include with a field of view that includes the electrode 106 and at least a portion of the tendon cut by the electrode 106 according to some embodiments. The angulation of the one or more cameras 124 can be between 5 degrees and 45 degrees relative to the longitudinal axis LA, for example.

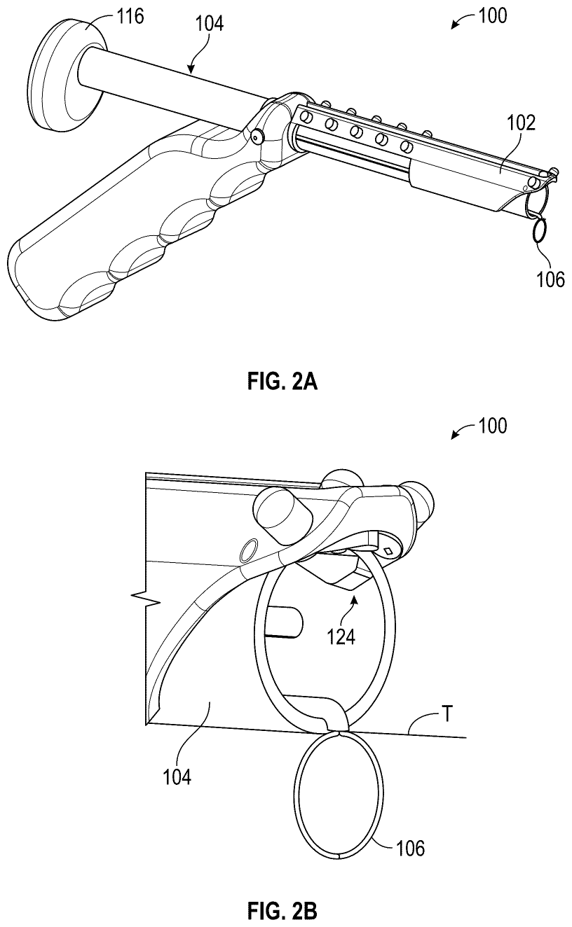

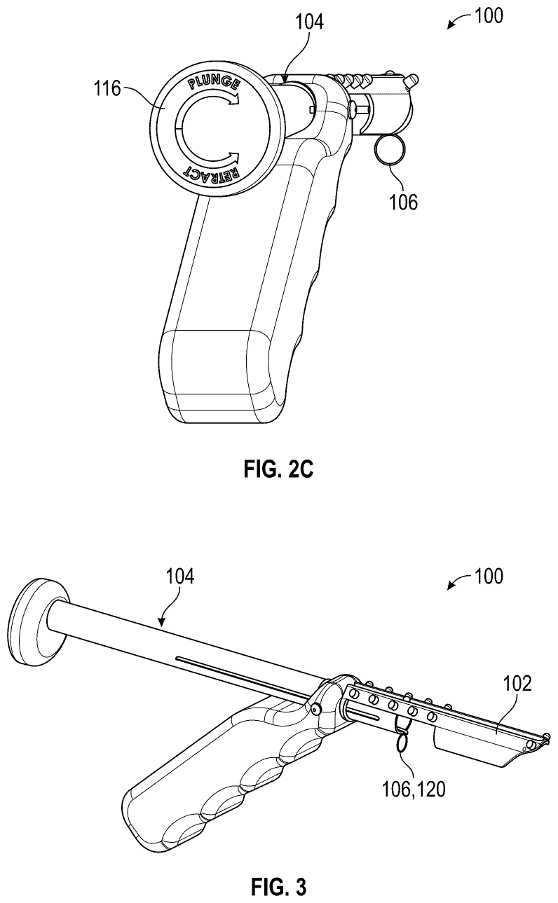

The surgical device 100 can be operatively coupled by a cable assembly or other mechanism to an energy source and/or a controller (not shown) which can control or aid with at least some of the functions such as visualization and RF implementation by the surgical device 100. The cable assembly can be coupled to various features including the RF electrode 106, the one or more cameras 124, etc. The controller, for example, can operate and control some or all functionality, which includes controlling the RF source, visualization using the one or more cameras 124, the illumination of the LEDs, etc. Additionally, the surgical device 100 can be operatively coupled to a fluid source for an irrigating fluid (e.g., saline). The irrigating fluid can be provided to the electrode 106 such as directly onto the electrode 106 via a hypo-tube (FIGS. 1D and 2B) to which the electrode 106 is secured at least partially inside of. The use of irrigating fluid improve the RF ablation performance of the loop electrode during graft cutting by reducing smoke, charring and eschar and increasing the surface area for the return current path. Suction for return of the irrigating fluid and ablated tissue, smoke, etc. can also be provided by the surgical device 100.

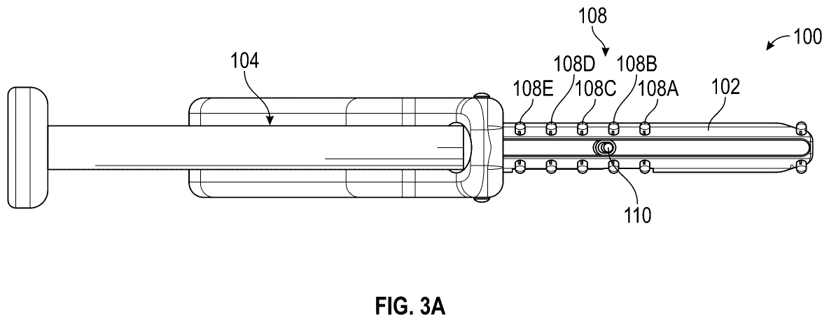

FIGS. 2A-3A show further operation of the surgical device 100. FIGS. 2A-2C show the electrode 106 actuated (rotated) by the actuation element 104 (the handle 116 of FIGS. 2A and 2B) to a plunge or initial deployed position. As shown in FIG. 2A, in this position the electrode 106 is activated and plunged down into the tendon T and this can be visualized by the one or more cameras 124. In the plunge or initial deployed position, the electrode 106 is external of the actuation element 104 and the housing 102. FIG. 2B shows the handle 116 rotated to the plunge position from the retracted position previously shown in FIG. 1C.

FIGS. 3 and 3A show the surgical device 100 with the actuation element 104 and the electrode 106 (not shown in FIG. 3A) moved to a completed tendon cutting position. In particular, the actuation element 104 and the electrode 106 have been moved proximally from the positions of FIGS. 1A-2C. This movement can include rotation of the electrode 106 to the initial deployed position (shown in FIGS. 2A-2C) and then linear movement of the actuation element 104 and the electrode 106 proximally relative to the housing 102. Such linear movement, along with application of the RF energy via the electrode 106 acting as the active electrode 120 to deliver RF current in a cutting waveform, creates a plasma that ablates the tendon to create the graft.

As shown in FIG. 3A, the actuation element 104 and the electrode 106 (shown in FIG. 3) are withdrawn proximally a distance while performing RF cutting. The first plurality of LEDs 108 and/or the second one or more LEDs 110 can be used as previously described in FIGS. 1A and 1B to provide surgeon with visualized location of where the surgical device 100 is located within the patient, the position of the electrode and to determine how long a length of the quadriceps tendon is being harvested. As the second one or more LEDs 110 move with the electrode 106 (FIG. 3) during actuation thereof back proximally toward the first plurality of LEDs 108A, 108B, 108C, 108D and 108E, the second one or more LEDs 110 allow the surgeon to judge the length of the graft being harvested in addition to the position of the electrode. As shown in FIG. 3A, the second one or more LEDs 110 can be generally aligned with one of the first plurality of LEDs 108A, 108B, 108C, 108D and 108E (here LED 108B) to obtain the desired length of tendon graft. Once cutting of the graft to the desired length is achieved, the RF energy can be deactivated, the electrode rotated back in the retracted position and the surgical device 100 can be removed from the patient. The graft can then be extracted from the patient using forceps or other surgical tools.

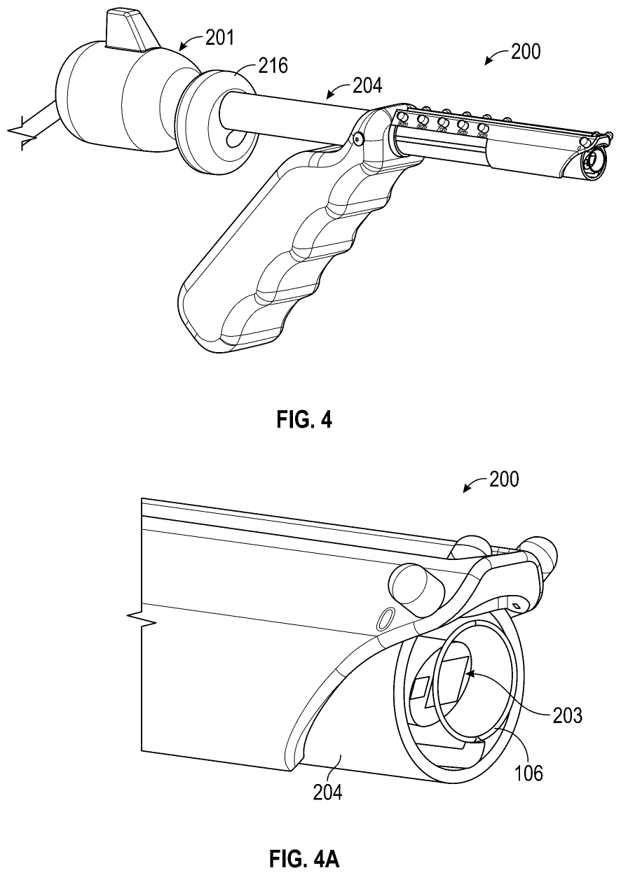

FIGS. 4 and 4A show a surgical device 200 according to another example. The surgical device 200 can be constructed in a manner similar to that of the surgical device 100 but can be configured to integrate with a second device 201 such as via a handle 216 (FIG. 4) and an actuation element 204. The second device 201 can be an endoscope having a visualization assembly 203 (FIG. 4A). The actuation element 204 can be hollow/tubular allowing the visualization assembly 203 to be positioned adjacent the electrode 106 (FIG. 4A). With the configuration of FIGS. 4 and 4A, the one or more cameras (shown in FIG. 1D) need not be integrated as part of the surgical device 200. Rather, the second device 201 (the endoscope) can be configured to provide camera(s) and/or illumination adjacent the electrode 106 (FIG. 4A). The second device 201 can be operably coupled to a fluid source for irrigating fluid. The irrigating fluid can be provided to the electrode 106 such as directly onto the electrode 106 via the second device 210 particularly by a passageway at the distal tip thereof adjacent the visualization assembly 203 (FIG. 4A) to which the electrode 106 is in close proximity. Suction for return of the irrigating fluid and ablated tissue, smoke, etc. can also be provided by the surgical device 200 or the second device 201.

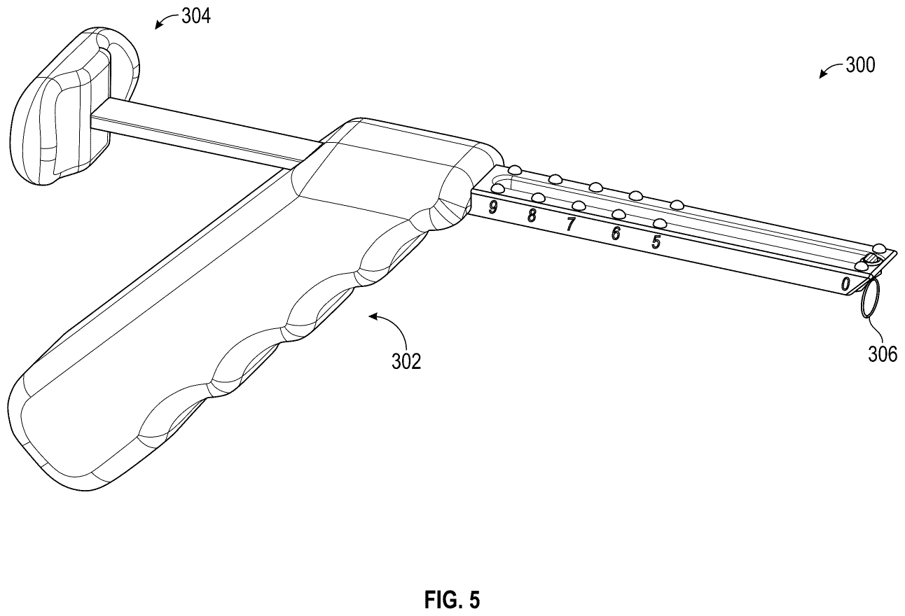

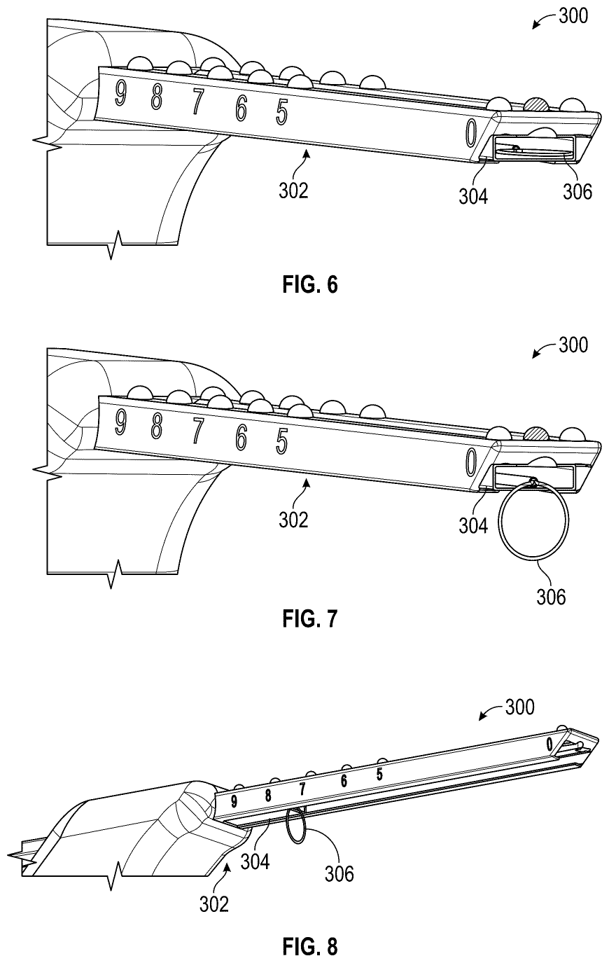

FIGS. 5-8 show a surgical device 300 according to another example. The surgical device 300 can be constructed in a similar manner to that of the surgical device 100 discussed previously and can include a housing 302, an actuation element 304 and an electrode 306. However, the surgical device 300 differs in construction from those shown previously in that the electrode 306 need not be rotated to deploy and is initially captured by the actuation element 304 in the insertion position shown in FIG. 6. The electrode 306 can be linearly translated distally out of the actuation element 304 and can be pre-bent to assume the plunge or initial deployed position (FIGS. 5 and 7) when freed from the actuation element 304. The electrode 306 can be a shape memory material (e.g., Nitinol or other suitable material(s)) and is configured to assume a pre-defined shape upon deployment to cut the tendon. FIG. 8 shows the electrode 306 and the actuation element 304 in a final cut position with the electrode 306 and the actuation element 304 moved proximally relative to the housing 302 to perform the cutting of the tendon in the manner described previously.

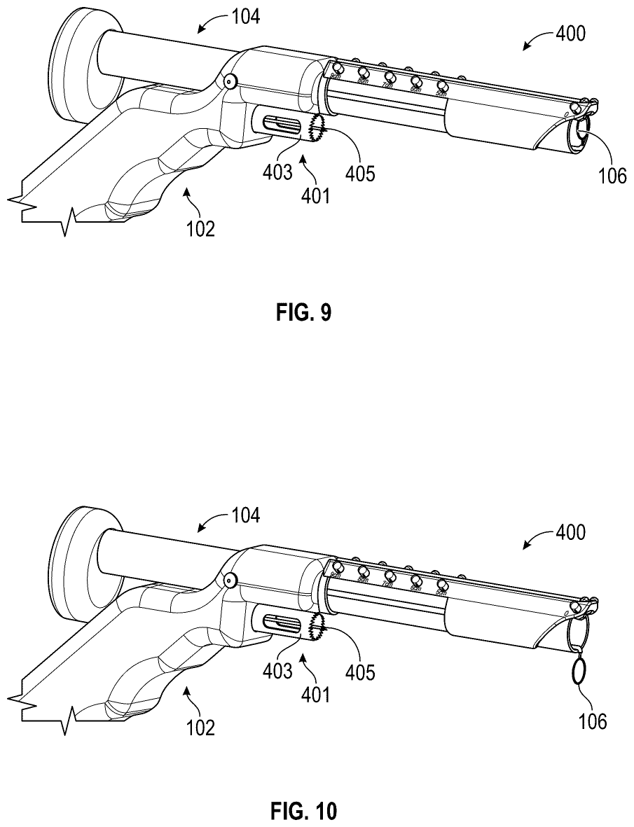

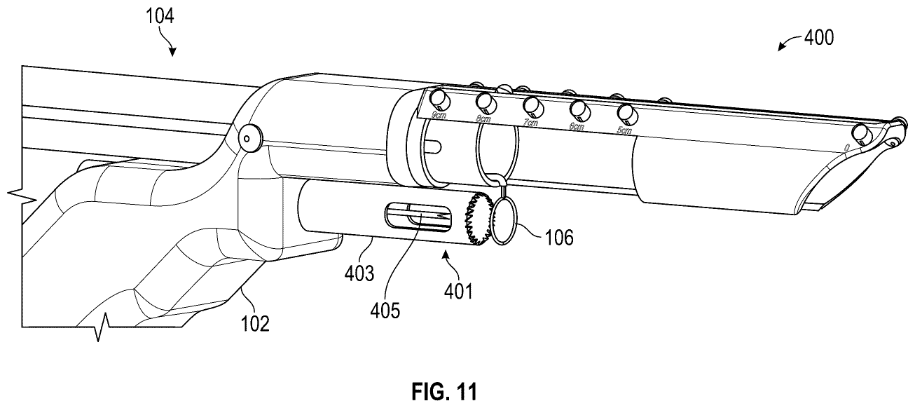

FIGS. 9-14 show another surgical device 400 constructed in a similar manner to the surgical device 100 having the housing 102, the actuation element 104, the electrode 106 but additionally including a bone cutting tool 401 configured to harvest a portion of a bone (e.g., a patella) connected to the graft. Thus, the surgical device 400 can be configured for harvesting a graft from a quadriceps tendon and a bone graft from the patella. The bone cutting tool 401 includes a bone corer 403 (e.g., a hole saw) and a center drill 405 (e.g., a stationary forked center pin). The bone cutting tool 401, in particular the bone corer 403, can be motor driven, for example. The FIGS. 9-11 capture the movement of the electrode 106 during the process of resecting the tendon minimally invasively. When the electrode 106 reaches the final cut position of FIG. 11, the surgeon can then activate a drive (e.g., a motor) to rotationally drive and linearly distally move the bone corer 403. The bone corer 403 can be arranged radially concentric with the loop of the electrode 106. This arrangement produces a bone plug at a diameter which generally matches the diameter of the graft of the tendon.

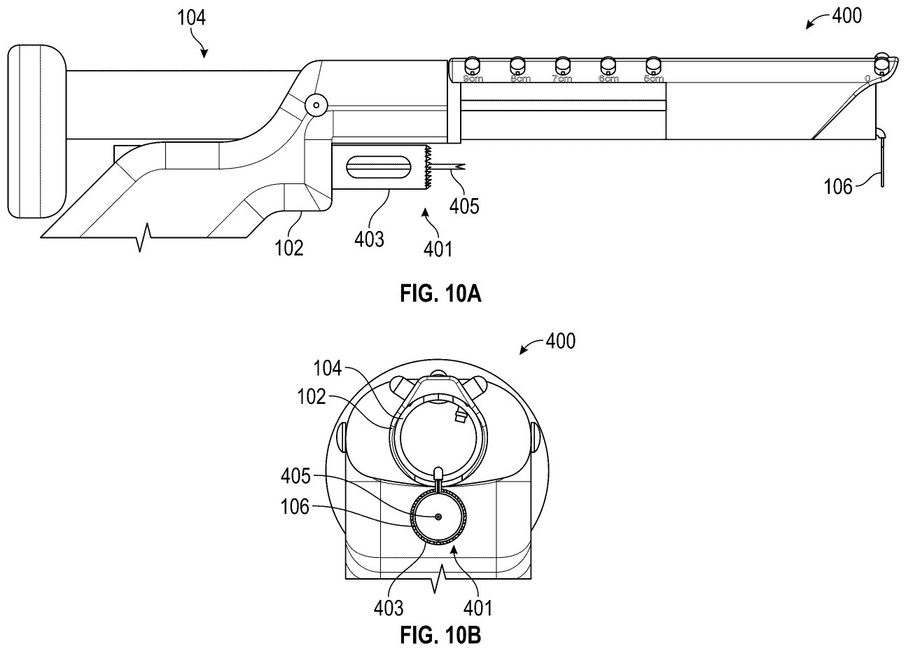

FIG. 9 shows the surgical device 400 with the electrode 106 in the insertion position at the distal end 122. FIGS. 10-10B shows the electrode 106 rotated to the initial insertion position (the plunge position) as discussed previously. For FIGS. 9-10B, the bone cutting tool 401 is not yet activated.

FIG. 11 shows the bone cutting tool 401 linearly moved distally to a final bone cutting position and the actuation element 104 and the electrode 106 moved proximally to a final tendon cutting position.

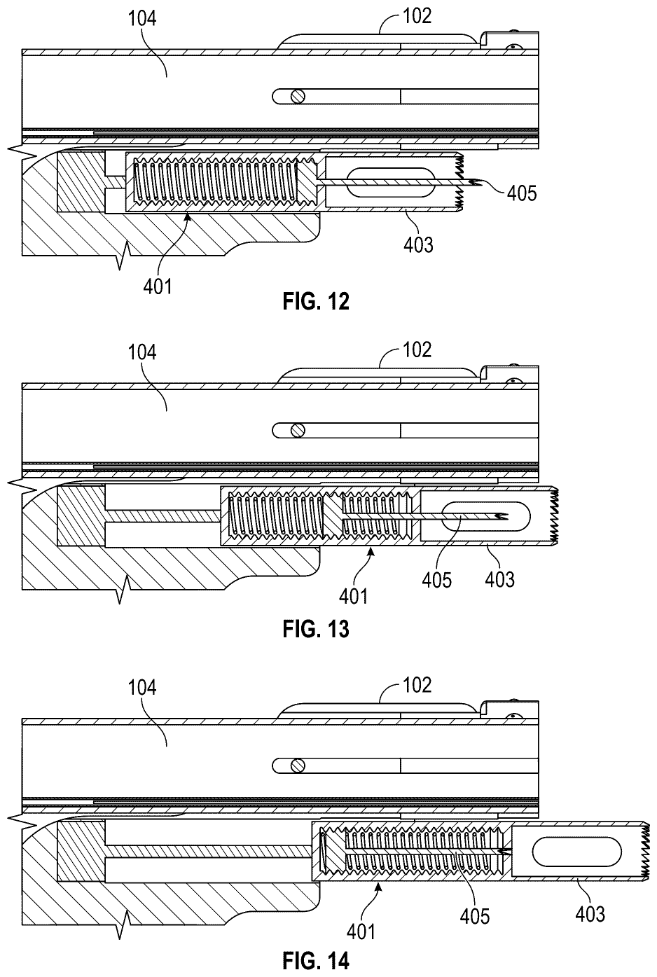

FIG. 12-14 show the process of actuation of the bone cutting tool 401 including the bone corer 403 and the center drill 405 according to one example. FIG. 12 shows the bone cutting tool 401 specifically with the bone corer 403 in a retracted position relative to the housing 102 and the actuation element 104. In FIG. 12, the center drill 405 extends from the bone corer 403 distally for initial contact/insertion in the bone. Throughout the obtaining of the bone graft, the center drill 405 can be configured to retain the portion of the bone for desired positioning and can prevent drift of the bone during harvesting of the portion of the bone for the bone graft.

Moving to FIG. 13, the bone corer 403 is partially extended linearly distally relative to the housing 102 and the actuation element 104. In FIGS. 12-14, the center drill 405 is threaded to remain substantially in a same initial contact position with the bone as shown in FIG. 12. Thus, the position of the center drill 405 does not change from FIG. 12 to FIG. 13 to FIG. 14 relative to the housing 102 and the actuation element 104.

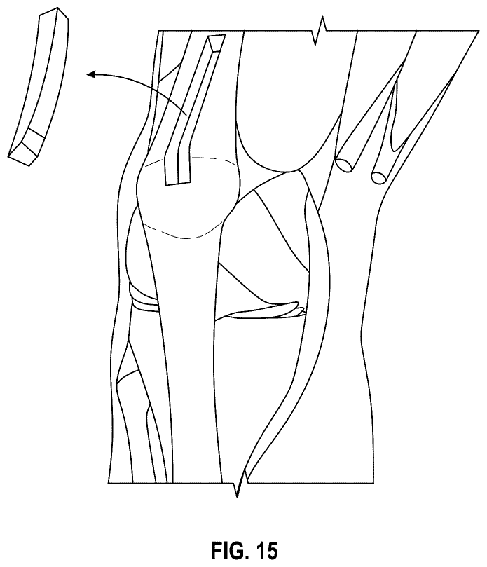

FIG. 14 shows the bone corer 403 in the fully extended final bone cutting position (also shown in FIG. 11) relative to the housing 102, the actuation element 104 and the center drill 405.

FIG. 15 schematically illustrates a bone graft and a tendon graft connected together that were obtained from the patella and the quadriceps tendon using the surgical device 400 of FIGS. 9-14.

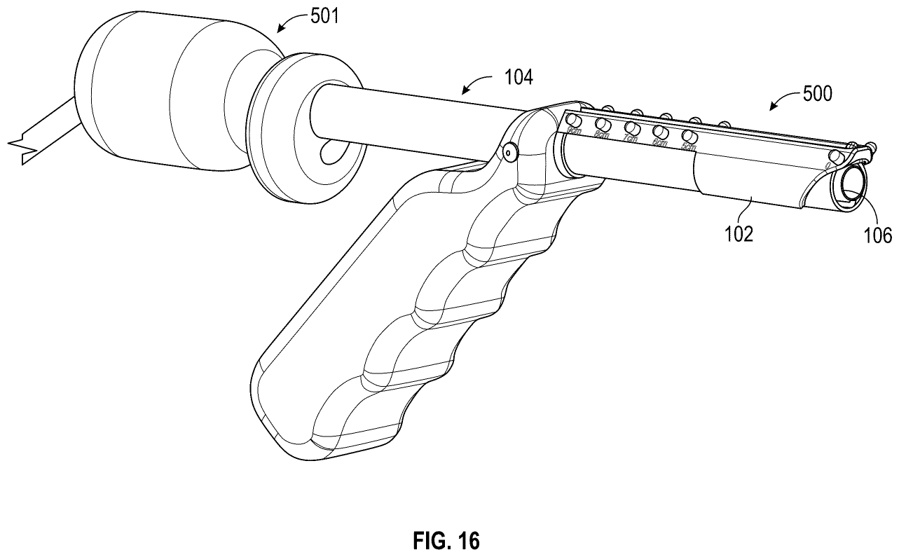

FIG. 16 show a surgical device 500 according to another example. The surgical device 500 can be constructed in a manner similar to that of the surgical device 200 but can be configured to integrate with a second device 501. The second device 501 can be a driver such motor configured to rotate and/or linearly move the first actuation element 104 and the electrode 106 relative to the housing 102 at a predetermined speed. This predetermined speed can be selected to reduce thermal injury to surrounding tissues.

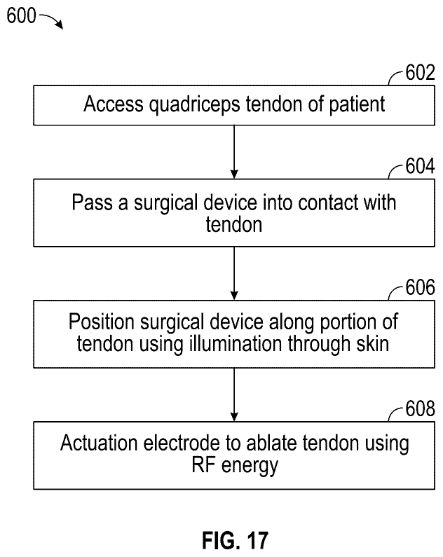

FIG. 17 illustrates a method 600 of harvesting a graft from a quadriceps tendon using a surgical device. The method 600 can optionally include accessing 602 the quadriceps tendon of a patient, passing 604 the surgical device into a contacting position along a portion of the quadriceps tendon, positioning 606 the surgical device along the portion of the quadriceps tendon to achieve a desired longitudinal length of the graft by illuminating one or more portions of the surgical device when inserted into the patient in the tendon contacting position and actuating 608 an electrode to cut the graft using radiofrequency (RF) energy.

Optionally, the method 600 can include visualizing the electrode with one or more cameras positioned adjacent the electrode. The actuating the electrode can include deploying the electrode from an initial insertion position where the electrode is protected by at least one of a housing or an actuation shaft of the surgical device. The actuating the electrode can include at least one of linear translation and rotation of the electrode relative to the housing or the actuation shaft. The method 600 can include harvesting a portion of a patella with a bone cutting tool of the surgical device, wherein the patella is connected to the graft. The positioning the surgical device to achieve the desired longitudinal length of the graft can include providing a proximal portion of the surgical device with a plurality of LEDs coupled thereto, wherein the plurality of LEDs are position indictive of various possible desired longitudinal lengths for the graft, wherein the plurality of LEDs are configured to produce illumination visible through skin of the patient when the surgical device is inserted into the patient. The positioning the surgical device to achieve the desired longitudinal length of the graft can include using one or more LEDs coupled for movement with the electrode, wherein the one or more LEDs are indicative of the position of the electrode and produce illumination visible through the skin of the patient when the surgical device is inserted into the patient.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. An electrosurgical device for harvesting a graft of a tendon comprising:
a housing having an elongate shape, the housing configured for insertion into an incision of a patient and is configured to contact the tendon upon insertion;
an actuation element moveable relative to the housing; and
an electrode coupled to the actuation element for movement therewith, wherein the electrode is configured to cut the tendon using radiofrequency (RF) energy.

2. The electrosurgical device of claim 1, wherein the electrosurgical device is a bipolar electrosurgical device with the electrode configured as an active electrode and the housing is configured as a return electrode, and wherein the return electrode serves as a return path for RF current.

3. The electrosurgical device of claim 1, wherein the electrode is configured as a loop electrode, and wherein the loop electrode is sized to cut the tendon to a desired lateral dimension.

4. The electrosurgical device of claim 1, wherein the electrode is a shape memory material and is configured to assume a pre-defined shape upon deployment to cut the tendon.

5. The electrosurgical device of claim 1, further comprising a plurality of light emitting diodes (LEDs), wherein the plurality of LEDs are position indictive of various desired longitudinal lengths for the graft, wherein the plurality of LEDs are configured to produce illumination visible through skin of the patient when the housing is inserted into the incision of the patient.

6. The electrosurgical device of claim 5, further comprising at least another LED coupled for movement with the electrode, wherein the at least another LED is indicative of the position of the electrode and produces illumination visible through the skin of the patient when the housing is inserted into the incision of the patient.

7. The electrosurgical device of claim 6, wherein the electrode is protected by at least one of the housing and the actuation element during initial insertion of the housing into the incision, and wherein the actuation element is configured to actuate the electrode to a plunge position external of the actuation element and the housing once inserted to a desired position.

8. The electrosurgical device of claim 7, wherein the actuation element is configured to actuate the electrode by at least one of linear translation and rotation of the electrode relative to the housing and a distal shaft of the actuation element.

9. The electrosurgical device of claim 1, further comprising one or more cameras coupled to the actuation element, wherein the one or more cameras are positioned with a field of view that includes the electrode and at least a portion of the tendon cut by the electrode.

10. The electrosurgical device of claim 1, further comprising a bone cutting tool configured to harvest a portion of a bone connected to the graft.

11. The electrosurgical device of claim 10, wherein the bone cutting tool includes a bone corer and a center drill configured to retain the portion of the bone for positioning and preventing drift during harvesting of the portion of the bone.

12. The electrosurgical device of claim 1, wherein the actuation element is configured to couple with a second device for one of visualization of the electrode or to drive actuation of the actuation element.

13. A method of harvesting a graft from a quadriceps tendon using a surgical device, comprising:
accessing the quadriceps tendon of a patient;
passing the surgical device into a contacting position along a portion of the quadriceps tendon;
positioning the surgical device along the portion of the quadriceps tendon to achieve a desired longitudinal length of the graft by illuminating one or more portions of the surgical device when inserted into the patient in the contacting position; and
actuating an electrode to cut the graft using radiofrequency (RF) energy.

14. The method of claim 13, further comprising visualizing the electrode with one or more cameras positioned adjacent the electrode.

15. The method of claim 13, wherein actuating the electrode includes deploying the electrode from an initial insertion position where the electrode is protected by at least one of a housing or an actuation shaft of the surgical device, wherein actuating the electrode includes at least one of linear translation and rotation of the electrode relative to the housing or the actuation shaft.

16. The method of claim 13, further comprising harvesting a portion of a patella with a bone cutting tool of the surgical device, wherein the patella is connected to the graft.

17. The method of claim 13, wherein positioning the surgical device to achieve the desired longitudinal length of the graft includes providing a proximal portion of the surgical device with a plurality of light emitting diodes (LEDs) coupled thereto, wherein the plurality of LEDs are position indictive of various possible desired longitudinal lengths for the graft, wherein the plurality of LEDs are configured to produce illumination visible through skin of the patient when the surgical device is inserted into the patient, wherein positioning the surgical device to achieve the desired longitudinal length of the graft includes using one or more LEDs coupled for movement with the electrode, wherein the one or more LEDs are indicative of the position of the electrode and produce illumination visible through the skin of the patient when the surgical device is inserted into the patient.

18. A bipolar electrosurgical device for harvesting a graft of a tendon comprising:
a return electrode configured to contact the tendon, wherein the return electrode is configured for insertion into an incision of a patient and is configured to contact the tendon upon insertion to provide a return path for radiofrequency (RF) current;
an actuation element moveable relative to the return electrode; and
an active electrode coupled to the actuation element for movement therewith, wherein the active electrode is configured to cut the tendon using the RF current.

19. The bipolar electrosurgical device of claim 18, wherein the active electrode is configured as a loop electrode, and wherein the loop electrode is sized to cut the tendon to a desired diameter, wherein the active electrode is protected by at least one of the return electrode and the actuation element during initial insertion of the return electrode into the incision, and wherein the actuation element is configured to actuate the active electrode to a plunge position external of the actuation element and the return electrode once inserted to a desired position.

20. The bipolar electrosurgical device of claim 18, wherein the active electrode is a shape memory material and is configured to assume a pre-defined shape upon deployment from one of the actuation element or the return electrode, wherein the return electrode includes a plurality of light emitting diodes (LEDs) coupled thereto, wherein the plurality of LEDs are position indictive of various desired longitudinal lengths for the graft, wherein the plurality of LEDs are configured to produce illumination visible through skin of the patient when a portion of the bipolar electrosurgical device is inserted into the incision of the patient, wherein the actuation element is configured to actuate the active electrode by at least one of linear translation and rotation of the active electrode relative to the return electrode and a distal shaft of the actuation element.
